(19) United States
(12) Patent Application Publication
(10) Pub. No.: US 2024/0173201 A1
(43) Pub. Date: May 30, 2024

(54) MANIPULATOR WITH HEATED MASSAGING FUNCTION

(71) Applicant: ZHEJIANG HAOZHONGHAO HEALTH PRODUCT CO., LTD., Wenzhou (CN)

(72) Inventors: Zhijing ZHOU, Wenzhou (CN); Yihe HUANG, Wenzhou (CN); Jiachen ZHOU, Wenzhou (CN)

(73) Assignee: ZHEJIANG HAOZHONGHAO HEALTH PRODUCT CO., LTD., Wenzhou (CN)

(21) Appl. No.: 18/283,034

(22) PCT Filed: Sep. 16, 2021
(86) PCT No.: PCT/CN2021/118713

§ 371 Date: Sep. 20, 2023

(30) Foreign Application Priority Data
Mar. 26, 2021 (CN) 202120622766.9

(51) Int. Cl. A61H 15/02; A61H 7/00; A61H 15/00

(52) U.S. Cl. CPC A61H 15/02; A61H 7/004; A61H 15/0078; A61H 2015/0014; A61H 2201/0149; A61H 2201/0207; A61H 2201/1215; A61H 2201/1623; A61H 2203/0431

(57) ABSTRACT

A manipulator a heated massaging function includes a main body. Two first massage assemblies are symmetrically arranged on two sides of the main body. The main body is provided with a driving mechanism and is further provided with a second massage assembly in a middle position between the two first massage assemblies. The second massage assembly includes a base, a first extension arm, a second extension arm, and at least two rolling heating assemblies. One end of the first extension arm and one end of the second extension arm are connected to the base in a swinging manner, and an elastic member is provided at a swing connection. The other end of the first extension arm and the other end of the second extension arm extend towards the upper and lower sides of the manipulator, respectively.

### CROSS REFERENCE TO THE RELATED APPLICATIONS

This application is the national phase entry of International Application No. PCT/CN2021/118713, filed on Sep. 16, 2021, which is based upon and claims priority to Chinese Patent Application No. 202120622766.9, filed on Mar. 26, 2021, the entire contents of which are incorporated herein by reference.

### TECHNICAL FIELD

The present disclosure relates to a manipulator with a heated massaging function.

### BACKGROUND

The Chinese patent application (No. CN112043565A) filed by the Applicant provides a manipulator with a heated massaging head. The manipulator includes a main body. Two first massage assemblies are symmetrically arranged on two sides of the main body. The main body is provided with a second massage assembly in a middle position between the two first massage assemblies. The second massage assembly includes a roller and a roller brace. The roller brace includes a roller mounting part matched with the roller and connected to the roller in a rolling manner, a base part matched and fixed with the main body, and an extension part connected to the roller mounting part and the base part. The extension part can swing towards the main body relative to the base part. An elastic member is provided between the extension part and the base part for the extension part to swing and reset. A heating mechanism is provided within the roller. When the manipulator moves along the guide rail of a massage chair, the structure above mentioned can drive the roller to keep close to multiple parts of the human body for rolling massage and adapt to the profile of the back of the human body. The heating mechanism in the roller can heat the roller, such that the roller can heat multiple parts of the human body while performing a rolling massage. The heating is concentrated and targeted, thus bringing the desired massage experience.

However, the manipulator has the following defects in actual use:
1. The second massage assembly can act on only the mid-back of the human body and cannot perform heated massaging on two sides of the back or the lower back.
2. The wire supplying power to the heating mechanism easily interferes with the massage assembly.

### SUMMARY

To overcome the shortcomings of the prior art, the present disclosure provides a manipulator with a heated massaging function, which has a more reasonable structure and a wider massage range.

To achieve the above objective, the present disclosure provides a manipulator with a heated massaging function. The manipulator is provided on a massage chair, movable along a surface of a human body, and includes a main body, where two first massage assemblies are symmetrically arranged on two sides of the main body; the main body is provided with a driving mechanism for driving the first massage assemblies; the main body is further provided with a second massage assembly in a middle position between the two first massage assemblies; the second massage assembly includes a base, a first extension arm and a second extension arm arranged on the base in a swinging manner, and at least two rolling heating assemblies respectively arranged on the first extension arm and the second extension arm; one end of the first extension arm and one end of the second extension arm are connected to the base in a swinging manner, and an elastic member is provided at a swing connection for the first extension arm and the second extension arm to swing and reset; the other end of the first extension arm and the other end of the second extension arm extend towards upper and lower sides of the manipulator, respectively; and the rolling heating assemblies are respectively located at the other end of the first extension arm and the other end of the second extension arm.

The present disclosure has the following beneficial effects. At least two rolling heating assemblies are movably arranged on the manipulator through the first extension arm and the second extension arm. Therefore, when the manipulator reciprocates along a guide rail of the massage chair, the force of the elastic member can keep the rolling heating assemblies tightly pressed on the back of the human body, thus improving the heating massage effect. Compared with the prior art, the present disclosure has a wider scope of action and stronger comfort.

Further, the other end of the first extension arm is provided with a laterally extended cross arm; a middle part of the cross arm is connected and fixed with the first extension arm; the other end of the first extension arm is provided with two of the rolling heating assemblies; the two rolling heating assemblies are respectively arranged at two ends of the cross arm; and the two rolling heating assemblies are respectively located above the two first massage assemblies.

The further design has the following effects. When the manipulator moves, the two rolling heating assemblies can act on two sides of the human spine at the same time, expanding the scope of action on the human body. In addition, the two rolling heating assemblies are located above the two first massage assemblies, which can just act on the motion path of the first massage assemblies to achieve rolling heating massage.

Further, the middle part of the cross arm is provided with a concave part; the concave part is provided with a cover plate; a chamber for wiring is formed between the cover plate and the concave part; and the chamber is provided with a through hole towards the one end of the first extension arm for a wire to pass through.

The further design has the following effects. The chamber can be used to accommodate the power supply wires of the rolling heating assemblies at the two ends of the cross arm and shield the wires to avoid interference between the wires and the rolling heating assemblies. In addition, the wires can be further extended towards the one end of the first extension arm through the through hole.

Further, the first extension arm and the second extension arm each are provided with a groove extending along two ends of the extension arm; and one end of the groove on the first extension arm corresponds to the through hole.

The further design has the following effects. The wire from the through hole of the chamber can be embedded in the groove and extended along the groove, such that the wire is hidden and protected to avoid interference between the wire and the moving part of the manipulator.

Further, the other end of the second extension arm is provided with one of the rolling heating assemblies; and the rolling heating assembly is located in the middle position between the two first massage assemblies.

The further design has the following effects. The rolling heating assembly at the other end of the second extension arm can massage the human spine when moving with the manipulator.

Further, the base is provided with a mounting table; two supporting parts are arranged on two sides of the mounting table; two groups of shaft holes are respectively arranged in the two supporting parts; the supporting parts each are provided with insertion ports for the shaft holes in a radial direction of the shaft holes; a shaft is inserted and positioned between the two groups of shaft holes; the one end of the first extension arm and the one end of the second extension arm are rotationally connected to the base through the shaft.

The further design has the following effects. The design facilitates the installation of the shaft and the connection between the first extension arm, the second extension arm, and the base.

Further, the base is provided with two limit sleeves for limiting rotation ranges of the first extension arm and the second extension arm, respectively; the two limit sleeves each include two supporting feet that are respectively matched and connected to the supporting parts of the base; and the two supporting feet of at least one of the limit sleeves can cover the insertion ports.

The further design has the following effects. When the limit sleeve is provided, the insertion ports can be covered to prevent the shaft from falling out, thus improving operation stability.

Further, multiple groups of positioning holes are symmetrically arranged on two sides of the groove.

The further design has the following effect. The positioning holes can be used to mount a wire clamp, a metal wire, a rope, or other fixing parts that can keep the wire in the groove to prevent the wire from shaking out.

### BRIEF DESCRIPTION OF THE DRAWINGS

FIG. 1 is a schematic diagram of a manipulator with a heated massaging function according to an embodiment of the present disclosure;

FIG. 2 is a side view of a second massage assembly according to an embodiment of the present disclosure;

FIG. 3 is an exploded view of the second massage assembly according to an embodiment of the present disclosure;

FIG. 4 is an assembly view of a first extension arm and a cross arm according to an embodiment of the present disclosure.

FIG. 5 is an assembly view of a second extension arm and a rolling heating assembly according to an embodiment of the present disclosure; and

FIG. 6 is a schematic diagram of a base according to an embodiment of the present disclosure.

### DETAILED DESCRIPTION OF THE EMBODIMENTS

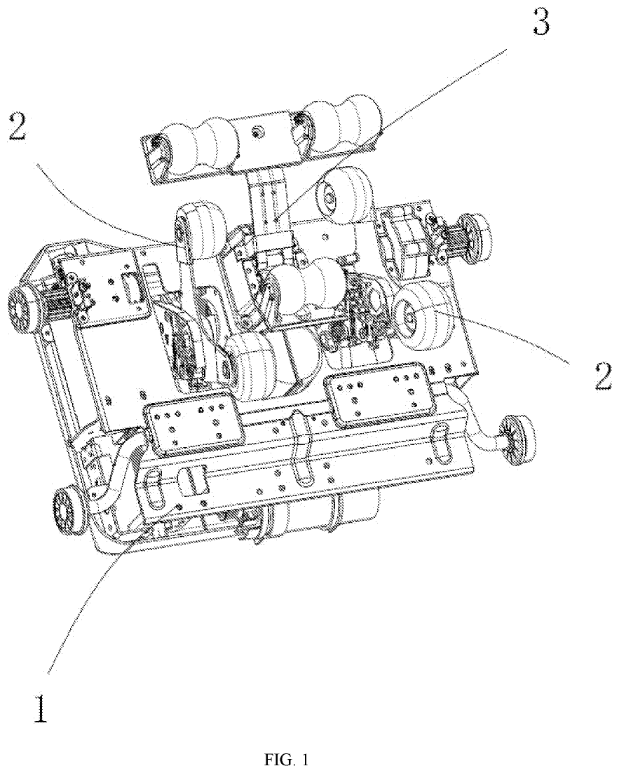

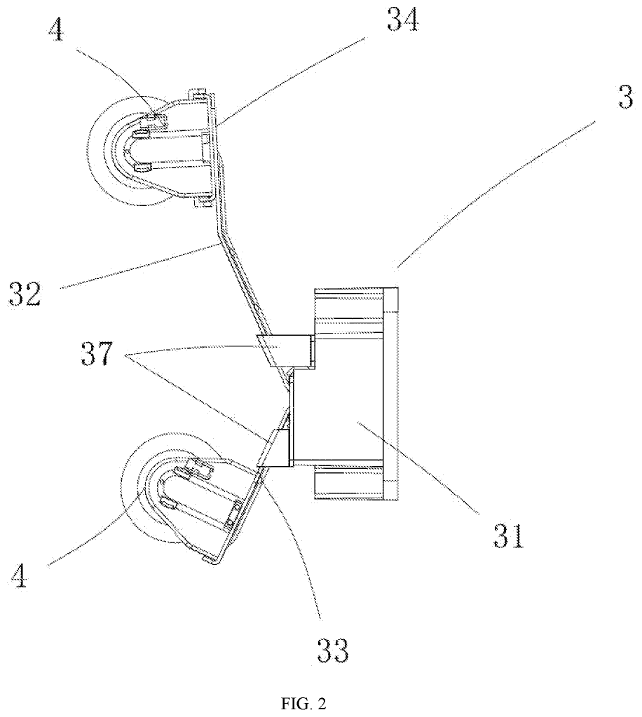

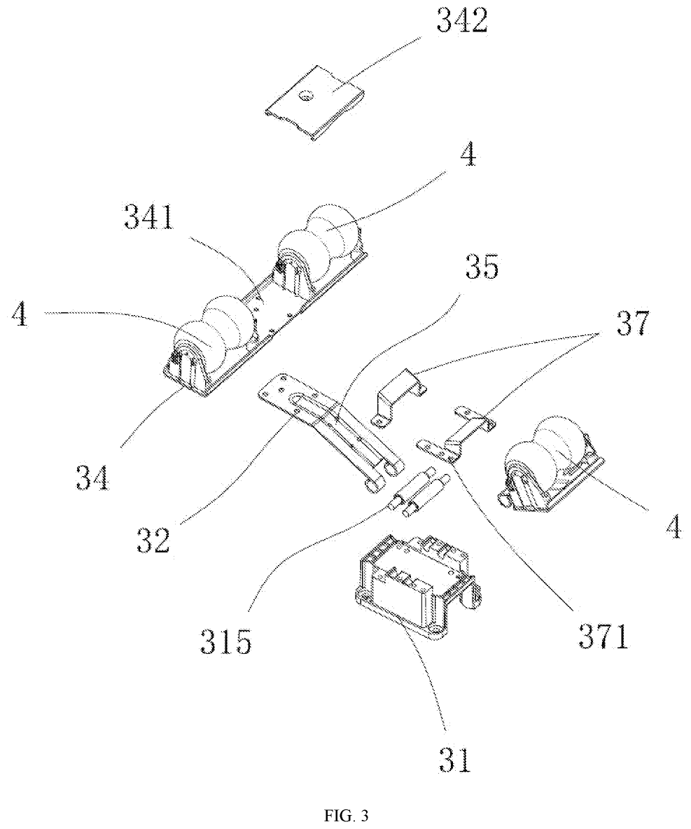

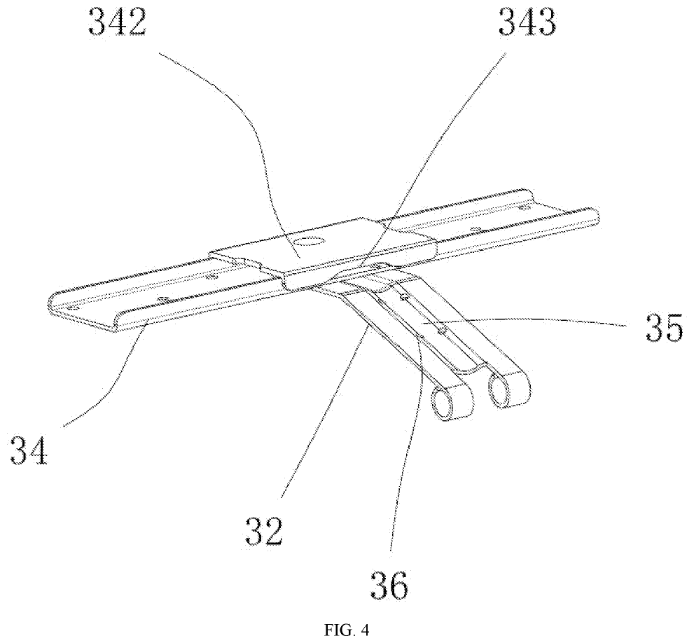

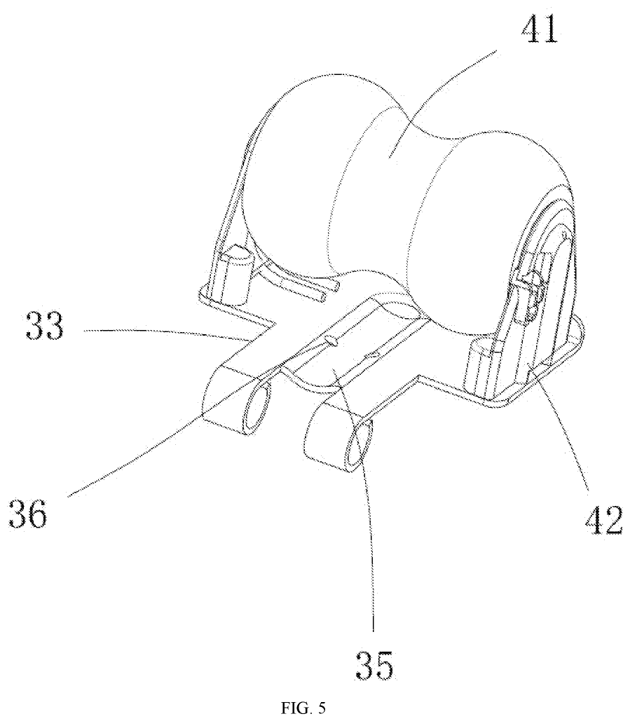

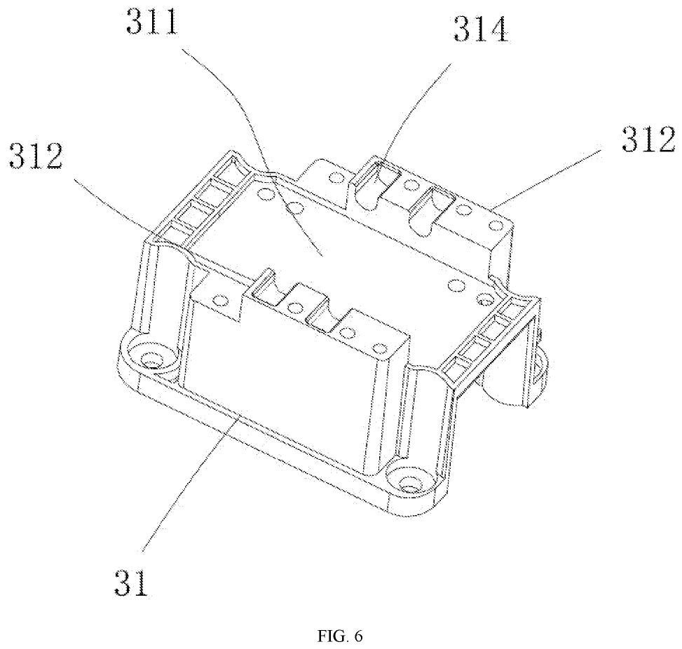

An embodiment of the present disclosure provides a manipulator with a heated massaging function. As shown in FIGS. 1 to 6, the manipulator is provided on a massage chair and is movable along the surface of the human body. The manipulator includes main body 1. Two first massage assemblies 2 are symmetrically arranged on two sides of the main body 1. The main body 1 is provided with a driving mechanism for driving the first massage assemblies 2. The driving mechanism takes at least one motor as a power source and can drive the first massage assemblies 2 to move to achieve massage actions such as kneading and knocking. The main body 1 is provided with the second massage assembly 3 in a middle position between the two first massage assemblies 2. The second massage assembly 3 makes reasonable use of the space and has little change to the structure of the conventional main body 1. The second massage assembly 3 includes base 31, first extension arm 32, and second extension arm 33 arranged on the base 31 in a swinging manner, and at least two rolling heating assemblies 4 respectively arranged on the first extension arm 32 and the second extension arm 33. The rolling heating assemblies 4 each include a heating module and a rolling ball, sphere, drum, or other similar rolling elements. The heating module can use a bulb or a heating wire. The heating module heats the rolling element to make the rolling element roll along the surface of the human body while heating a local part of the human body to achieve the effects of massage, hot compress, physical therapy, etc. In this embodiment, the rolling heating assemblies 4 each include dumbbell-shaped roller 41, brace 42 rotationally connected at two ends of the roller 41, and a heating module provided inside the roller 41. The brace 42 rotationally fixes the roller 41 on the first extension arm 32 or the second extension arm 33. One end of the first extension arm 32 and one end of the second extension arm 33 are connected to the base 31 in a swinging manner. An elastic member is provided at a swing connection for the first extension arm 32 and the second extension arm 33 to swing and reset. The elastic member provides an elastic force that keeps the rolling heating assemblies 4 pressed on the surface of the human body to improve the contact effect between the rolling heating assemblies and the surface of the human body. The other end of the first extension arm 32 and the other end of the second extension arm 33 extend towards the upper and lower sides of the manipulator, respectively. The rolling heating assemblies 4 are respectively located at the other end of the first extension arm 32 and the other end of the second extension arm 33. Rolling axes of all the rolling heating assemblies 4 are parallel to each other to reduce interference when rolling along the surface of the human body, thus improving comfort.

The other end of the first extension arm 32 is provided with a laterally extended cross arm 34. The middle part of the cross arm 34 and the other end of the first extension arm 32 are connected and fixed by a bolt. Of course, they can also be directly integrated. The other end of the first extension arm 32 is provided with the two rolling heating assemblies 4. The two rolling heating assemblies 4 are respectively arranged at two ends of the cross arm 34. The two rolling heating assemblies 4 are respectively located above the two first massage assemblies 2.

The middle part of the cross arm 34 is provided with concave part 341. The concave part 341 is provided with cover plate 342. A chamber for wiring is formed between the cover plate 342 and the concave part 341. The chamber is provided with through hole 343 for wires to pass through towards the one end of the first extension arm 32. The chamber is located between two rolling heating assemblies 4 on the first extension arm 32. The wires of the two rolling heating assemblies 4 are arranged together in the chamber and extend along the one end of the first extension arm 32 through the through hole 343.

The first extension arm 32 and the second extension arm 33 each are provided with a groove 35 extending along two ends of the extension arm. One end of the groove 35 on the first extension arm 32 corresponds to the through hole 343, which is convenient for the arrangement of the wires. Multiple groups of positioning holes 36 are symmetrically arranged on two sides of groove 35. The positioning holes 36 can be used to mount a wire clamp, a metal wire, a rope, or other fixing part that can keep the wire in the groove 35 to prevent the wire from shaking out.

The other end of the second extension arm 33 is provided with the rolling heating assembly 4. The rolling heating assembly 4 is located in the middle position between the two first massage assemblies 2.

The base 31 is provided with mounting table 311. Two supporting parts 312 are arranged on two sides of the mounting table 311. Two groups of shaft holes 313 are respectively arranged in the two supporting parts 312. The supporting parts 312 each are provided with insertion ports 314 for the shaft holes 313 in a radial direction of the shaft holes 313. A shaft 315 is inserted between the two groups of shaft holes 313. The one end of the first extension arm 32 and the one end of the second extension arm 33 are rotationally connected to the base 31 through the shaft 315. The shaft 315 is sleeved with a shaft sleeve. The one end of the first extension arm 32 and the one end of the second extension arm 33 each are provided with a sleeve hole sleeved on the shaft sleeve. The design can reduce friction and wear during rotation.

The base 31 is provided with two limit sleeves 37 for limiting rotation ranges of the first extension arm 32 and the second extension arm 33 respectively. The limit sleeves 37 limit the rotation ranges of the first extension arm 32 and the second extension arm 33 within 90 degrees.

The two limit sleeves 37 each include two supporting feet 371 that are respectively matched and connected to the supporting parts 312 of the base 31. The two supporting feet 371 of at least one of the limit sleeves 37 can cover the insertion ports 314 to prevent two ends of the shaft 315 from coming out. The elastic member (not shown in the figure) can be a torsion spring sleeved on the shaft 315, a tension spring, or a compression spring. The elastic member provides an elastic force to maintain the other end of the first extension arm 32 and the other end of the second extension arm 33 at a swing angle farthest from the main body 1.

The above embodiment is just one of the preferred specific embodiments of the present disclosure, and normal variations and substitutions made by those skilled in the art within the scope of the technical solution of the present disclosure should fall within the protection scope of the present disclosure.

What is claimed is:

1. A manipulator with a heated massaging function, provided on a massage chair, movable along a surface of a human body, and comprising a main body, wherein
two first massage assemblies are symmetrically arranged on two sides of the main body; the main body is provided with a driving mechanism for driving the two first massage assemblies; the main body is further provided with a second massage assembly in a middle position between the two first massage assemblies;
the second massage assembly comprises a base, a first extension arm, a second extension arm, and at least two rolling heating assemblies, wherein the first extension arm and the second extension arm are arranged on the base in a swinging manner, and the at least two rolling heating assemblies are respectively arranged on the first extension arm and the second extension arm;
a first end of the first extension arm and a first end of the second extension arm are connected to the base in a swinging manner, and an elastic member is provided at a swing connection for the first extension arm and the second extension arm to swing and reset;
a second end of the first extension arm and a second end of the second extension arm extend towards upper and lower sides of the manipulator, respectively; and
the at least two rolling heating assemblies are respectively located at the second end of the first extension arm and the second end of the second extension arm.

2. The manipulator according to claim 1, wherein the second end of the first extension arm is provided with a cross arm laterally extending; a middle part of the cross arm is connected and fixed with the first extension arm; the second end of the first extension arm is provided with two rolling heating assemblies; the two rolling heating assemblies are respectively arranged at two ends of the cross arm; and the two rolling heating assemblies are respectively located above the two first massage assemblies.

3. The manipulator according to claim 2, wherein the middle part of the cross arm is provided with a concave part; the concave part is provided with a cover plate; a chamber for wiring is formed between the cover plate and the concave part; and the chamber is provided with a through hole towards the first end of the first extension arm and a wire passes through the through hole.

4. The manipulator according to claim 3, wherein each of the first extension arm and the second extension arm is provided with a groove extending along two ends of the first extension arm and the second extension arm; and a first end of the groove on the first extension arm corresponds to the through hole.

5. The manipulator according to claim 1, wherein the second end of the second extension arm is provided with one of the at least two rolling heating assemblies; and the one rolling heating assembly is located in the middle position between the two first massage assemblies.

6. The manipulator with the heated massaging function according to claim 1, wherein the base is provided with a mounting table; two supporting parts are arranged on two sides of the mounting table; two groups of shaft holes are respectively arranged in the two supporting parts; the two supporting parts each are provided with insertion ports in a radial direction of the shaft holes; a shaft is inserted and positioned between the two groups of shaft holes; and the first end of the first extension arm and the first end of the second extension arm are rotationally connected to the base through the shaft.

7. The manipulator according to claim 6, wherein the base is provided with two limit sleeves for limiting rotation ranges of the first extension arm and the second extension arm, respectively; each of the two limit sleeves comprises two supporting feet, and the two supporting feet are respectively matched and connected to the supporting parts of the base; and the two supporting feet of at least one of the two limit sleeves is configured to cover the insertion ports.

8. The manipulator according to claim 4, wherein multiple groups of positioning holes are symmetrically arranged on two sides of the groove.

9. The manipulator according to claim 2, wherein the base is provided with a mounting table; two supporting parts are arranged on two sides of the mounting table; two groups of shaft holes are respectively arranged in the two supporting parts; the two supporting parts each are provided with insertion ports in a radial direction of the shaft holes; a shaft is inserted and positioned between the two groups of shaft holes; and the first end of the first extension arm and the first end of the second extension arm are rotationally connected to the base through the shaft.

10. The manipulator according to claim 3, wherein the base is provided with a mounting table; two supporting parts are arranged on two sides of the mounting table; two groups of shaft holes are respectively arranged in the two supporting parts; the two supporting parts each are provided with insertion ports in a radial direction of the shaft holes; a shaft is inserted and positioned between the two groups of shaft holes; and the first end of the first extension arm and the first end of the second extension arm are rotationally connected to the base through the shaft.

11. The manipulator according to claim 4, wherein the base is provided with a mounting table; two supporting parts are arranged on two sides of the mounting table; two groups of shaft holes are respectively arranged in the two supporting parts; the two supporting parts each are provided with insertion ports in a radial direction of the shaft holes; a shaft is inserted and positioned between the two groups of shaft holes; and the first end of the first extension arm and the first end of the second extension arm are rotationally connected to the base through the shaft.

12. The manipulator according to claim 5, wherein the base is provided with a mounting table; two supporting parts are arranged on two sides of the mounting table; two groups of shaft holes are respectively arranged in the two supporting parts; the two supporting parts each are provided with insertion ports in a radial direction of the shaft holes; a shaft is inserted and positioned between the two groups of shaft holes; and the first end of the first extension arm and the first end of the second extension arm are rotationally connected to the base through the shaft.
